(19) United States
(12) Patent Application Publication
(10) Pub. No.: US 2021/0283237 A1
(43) Pub. Date: Sep. 16, 2021

(54) VACCINE COMPOSITIONS

(71) Applicant: Emergex Vaccines Holding Limited, Oxfordshire (GB)

(72) Inventors: Laurens Rademacher, Oxfordshire (GB); Thomas Rademacher, Oxfordshire (GB); Ramila Philip, Sparks, NV (US)

(21) Appl. No.: 16/982,036

(22) PCT Filed: Mar. 29, 2019
(86) PCT No.: PCT/GB2019/050929

§ 371 Date: Sep. 17, 2020

Related U.S. Application Data
(60) Provisional application No. 62/649,812, filed on Mar. 29, 2018.

(51) Int. Cl. A61K 39/12; A61K 47/69; A61P 31/14; A61K 9/16; C12N 7/00

(52) U.S. Cl. CPC A61K 39/12; A61K 47/6923; A61P 31/14; A61K 2039/60; A61K 9/1611; A61K 9/1623; C12N 7/00; A61K 9/167

(57) ABSTRACT

The invention provides a vaccine composition comprising a filovirus peptide comprising one or more CD8+ T cell epitopes, wherein the peptide is attached to a nanoparticle

### FIELD OF THE INVENTION

The invention relates to vaccine compositions comprising filovirus peptides, and the use of such compositions for the treatment and prevention of filovirus infection.

### BACKGROUND TO THE INVENTION

Filoviruses including marburgviruses and ebolaviruses pose a significant threat to public health. In particular, marburgviruses and ebolaviruses cause severe haemorrhagic fevers with high mortality in humans and non-human primates. Filoviral haemorrhagic fevers are characterised by a syndrome resembling septic shock, which results from a combination of pathogenic mechanisms that enables the virus to suppress innate and adaptive immune responses, infect and kill a variety of cell types, and induce strong inflammatory responses and disseminated intravascular coagulation.

Ebolavirus was first discovered in 1976 near the Ebola River in what is now the Democratic Republic of the Congo. Marburgvirus was first described in the 1960s in Germany, following the exposure of German workers to tissues of infected monkeys. Outbreaks of haemorrhagic fever associated with both filoviruses have since appeared sporadically in Africa, where fruit bats are suspected to be their natural reservoir. However, the mode of primary transmission to humans remains evasive. Transmission between humans appears to require close proximity, such as direct contact with blood or other bodily fluids leading to mucous membrane exposure, or the handling of fomites. Several studies have though indicated that filoviruses may be capable of aerosol transmission in the laboratory, and there are fears that airborne spread among humans may be possible or become possible as viruses evolve.

Currently, no specific treatment or prophylaxis for filovirus infection is approved for clinical use in humans. Treatment is generally supportive, and barrier nursing techniques are implemented to minimise transmission between humans. Such techniques are not, of course, fail safe, and would be less effective should filoviruses become capable of aerosol transmission. A vaccine against filovirus infection is therefore an attractive prospect.

In this regard, some success has been achieved with recombinant vesicular stomatitis virus-Zaire Ebola virus (rVSV-ZEBOV), an experimental vaccine for protection against Ebola virus disease. Ring vaccination with rVSV-ZEBOV appears to be somewhat effective, but the efficacy of the vaccine is still uncertain. Furthermore, around 1 in 2 people given the rVSV-ZEBOV vaccine experience mild to moderate adverse effects that include headache, fatigue and muscle pain. This is perhaps unsurprising given that the rVSV-ZEBOV vaccine is a recombinant, replication competent vesicular stomatitis virus (VSV), and that VSV leads to a flu-like illness in infected humans.

The VSV comprised in the rVSV-ZEBOV vaccine is genetically modified to express a glycoprotein from the Zaire ebolavirus (ZEBOV), so as to elicit a neutralizing immune response against the ebolavirus. It is not clear, however, whether vaccination with rVSV-ZEBOV protects against infection with ebolaviruses other than the Zaire ebolavirus species (such as Bundibugyo ebolavirus (BDBV), Reston ebolavirus (RESTV), Sudan ebolavirus (SUDV), and Taï Forest ebolavirus (TAFV)), or other types of filoviruses (such as the marburgviruses Marburg virus (MARV) and Ravn virus (RAVV)). It is also not clear whether vaccination with rVSV-ZEBOV would protect against emerging ebolavirus strains.

A vaccine providing broad protection against multiple marburgvirus and/or ebolavirus species, or indeed multiple filoviruses, is desirable. This would permit swift ring vaccination in the case of an outbreak of haemorrhagic fever without the need to first determine the particular virus involved in the disease. Such a vaccine could also be used at the population level to induce widespread immunity to filoviruses, guarding against future outbreaks. Furthermore, administration of a single cross-protective vaccine may help to limit the costs associated with vaccine production and dissemination, allowing cost-effective prophylaxis in the developing countries where ebolavirus infection and marburgvirus infection are most prevalent. Cross-protection against emerging filoviruses may also prevent the establishment of filoviruses capable of airborne spread.

### SUMMARY OF THE INVENTION

The present invention relates to a filovirus vaccine composition that stimulates an immune response while avoiding the adverse clinical effects often associated with vaccines containing viruses. The vaccine composition may provide protection against multiple genera of filovirus (e.g. ebolavirus and marburgvirus) and/or multiple species of ebolavirus and/or marburgvirus.

The present inventors have surprisingly identified that a nanoparticle, for example a gold nanoparticle, may be used to induce an efficient response to a vaccine composition designed to stimulate a T cell response against filovirus. Use of a nanoparticle abrogates the need to use a virus in the vaccine composition. The use of a traditional adjuvant, which may be associated with adverse reactions in the clinic, is also avoided. Therefore, the likelihood of an individual experiencing an adverse reaction following administration of the vaccine composition is reduced.

The present inventors have also identified number of peptides that are conserved between different filoviruses and are presented by MHC molecules on cells infected with those viruses. Inclusion of such conserved peptides in the vaccine composition may confer protective capability against multiple genera of filovirus, and/or multiple species of ebolavirus and/or marburgvirus. Including multiple conserved peptides that bind to different HLA supertypes in the vaccine composition results in a vaccine that is effective in individuals having different HLA types.

Accordingly, the present invention provides a vaccine composition comprising a filovirus peptide comprising one or more of the CD8+ T cell epitopes set out in SEQ ID NOs: 1 to 9, wherein the peptide is attached to a nanoparticle.

The present invention further provides:
a method of preventing or treating a filovirus infection, comprising administering the vaccine composition of any one of the preceding claims to an individual infected with, or at risk of being infected with, a filovirus; and
a vaccine composition of the invention for use in a method of preventing or treating a filovirus infection in an individual.

### BRIEF DESCRIPTION OF THE FIGURES

FIG. 1: Activated PBMCs were analysed for levels of CD8 to determine expansion of this population of cells. All 6 peptides caused (P1-6) cell expansion. Non-loaded T2 cells (T2) acted as control. A high level of non-peptide specific expansion was observed. The error bars represent the standard deviation of the replicates.

FIG. 2: The CD107a expression from CD8+ population of PBMCs after activation with peptide loaded T2s. All 6 peptide (P1-6) epitopes activated CD8+ T cells to respond to peptide loaded T2 cells in a Filovirus peptide specific manner. The error bars represent the standard deviation of the replicates peptide specific manner. Non-loaded T2 cells (T2) acted as control.

FIG. 3: The IFNγ expression from CD8+ population of PBMCs after activation with peptide loaded T2s. All 6 peptide (P1-6) epitopes activated CD8+ T cells to respond to peptide loaded T2 cells in a Filovirus peptide specific manner. Non-loaded T2 cells (T2) acted as control. The error bars represent the standard deviation of the replicates.

FIG. 4: Activated PBMCs were analysed for levels of CD8 to determine expansion of this population of cells. Free peptides (FP) and AuNP peptide conjugates (AuNP+peptide) were tested. All 6 AuNP peptide conjugates caused cell expansion comparable, albeit moderately lower than, free peptides. The bare AuNP particle did induce some cell expansion as expected.

FIG. 5: The CD107a expression from CD8+ population of PBMCs after activation with peptide loaded T2s. Free peptides (FP) and AuNP peptide conjugates (AuNP+peptide) were tested. Bare AuNP (AuNP) base particle acted as a control. All 6 AuNP peptide conjugates activated CD8+ T cells to respond to peptide loaded T2 cells in a Filovirus peptide specific manner comparable to the free peptides. The bare AuNP particle did induce some cell expansion as expected.

FIG. 6: The IFNγ expression from CD8+ population of PBMCs after activation with peptide loaded T2s. Free peptides (FP) and AuNP peptide conjugate (AuNP+peptide) were tested. Bare AuNP (AuNP) base particle acted as a control. All 6 AuNP peptide conjugates activated CD8+ T cells to respond to peptide loaded T2 cells in a Filovirus peptide specific manner comparable, albeit moderately lower than, free peptides. The bare AuNP particle did induce some cell expansion as expected.

FIG. 7: The cytokine expression analysed from supernatants of activated PBMCs against peptide loaded targets. Cytokines analysed include, IFN-g, IL-10, Granzyme B, TNF-α, and Perforin. Free peptides (FP) and AuNP peptide conjugate (AuNP+peptide) were tested. Bare AuNP (AuNP) base particle acted as a control. IFH-g, IL-10 and Granzyme B, all showed superior expression with AuNP peptide conjugates as compared to free peptides. TNFα expression was equivalent and perforin expression was decreased. The bare AuNP particle did induce some cell expansion as expected.

FIG. 8: The CD107a expression from CD8+ population of PBMCs after activation with peptide loaded HepG2 cells. Nanoparticle-conjugated P1, nanoparticle-conjugated P6, nanoparticle-conjugated P8, nanoparticle-conjugated P9, and pooled nanoparticle-conjugated P1, P6, P7, P8 and P9 all induced CD8+CD107a expression to peptide loaded HepG2 cells in a Filovirus peptide specific manner. The error bars represent the standard deviation of the replicates. Non-loaded HepG2 cells acted as control.

### DETAILED DESCRIPTION OF THE INVENTION

### Vaccine Compositions

The present invention provides a vaccine composition comprising a filovirus peptide comprising one or more of the CD8+ T cell epitopes set out in SEQ ID NOs: 1 to 9, wherein the peptide is attached to a nanoparticle. This vaccine composition has a number of advantageous over filovirus vaccines known in the art. The key advantages are summarised here. However, further advantages will become apparent from the discussion below.

Firstly, the vaccine composition of the invention advantageously comprises a filovirus peptide comprising one or more of the CD8+ T cell epitopes set out in SEQ ID NOs: 1 to 9. The vaccine composition is therefore capable of stimulating a cellular immune response against a filovirus. CD8+ cytotoxic T lymphocytes (CTLs) mediate viral clearance via their cytotoxic activity against infected cells. Stimulating cellular immunity may therefore provide a beneficial first-line defense against filovirus infection.

Secondly, the filovirus peptide in the vaccine composition is attached to a nanoparticle, for example a gold nanoparticle. As described in more detail below, attachment to a nanoparticle reduces or eliminates the need to include an adjuvant in the vaccine composition. Thus, the vaccine composition is less likely to cause adverse clinical effects upon administration to an individual.

### Peptides

The vaccine composition of the invention comprises a filovirus peptide comprising one or more of the CD8+ T cell epitopes set out in SEQ ID NOs: 1 to 9. The vaccine composition may comprise from about one to about 50 such peptides, such as about 2 to 40, 3 to 30, 4 to 25, 5 to 20, 6 to 15, 7, 8, 9 or 10 such peptides. SEQ ID NOs: 1 to 9 are set out in Table 1.

TABLE 1

| SEQ | | Pro- | | | |
|---|---|---|---|---|---|
| ID | | tein | HLA | Conserved | |
| NO: | Sequence | ID | affinity | between | Origin |
| 1 | KIIKFLEPL | L-293 | a2/24 | EBOV, SUDV, | ZEBOV |
| | | | | BDBV, RESTV, | infected |
| | | | | TAFV, MARV | cells |
| 2 | GLFQLKTYL | L-932 | a2 | EBOV, SUDV, | ZEBOV |
| | | | | BDBV, RESTV, | infected |
| | | | | TAFV | cells |
| 3 | VLKAVVLKV | L-1955 | a2 | EBOV, SUDV, | ZEBOV |
| | | | | BDBV, RESTV, | infected |
| | | | | TAFV | cells |
| 4 | RLAKLTEAI | NP-401 | a2 | EBOV, SUDV, | ZEBOV |
| | | | | BDBV, RESTV, | infected |
| | | | | TAFV | cells |
| 5 | IIQAFEAGV | NP-56 | a2 | EBOV, SUDV, | ZEBOV |
| | | | | BDBV, RESTV, | infected |
| | | | | TAFV | cells |
| 6 | KYTMQDALF | L-105 | a24 | EBOV, SUDV, | ZEBOV |
| | | | | RESTV, MARV | infected |
| | | | | | cells |
| 7 | KYQVKTLFF | L-1847 | a24 | EBOV, SUDV, | ZEBOV |
| | | | | BDBV, RESTV, | infected |
| | | | | TAFV | cells |
| 8 | QYADCELHL | L-2046 | a24 | EBOV, BDBV, | ZEBOV |
| | | | | TAFV, MARV | infected |
| | | | | | cells |
| 9 | SLTDRELLL | VP30-94 | a2/24 | EBOV, SUDV, | ZEBOV |
| | | | | BDBV, RESTV, | infected |
| | | | | TAFV, MARV | cells |

The filovirus peptide comprising one or more of the CD8+ T cell epitopes set out in SEQ ID NOs: 1 to 9 may comprise only one of the CD8+ T cell epitopes set out in SEQ ID NOs: 1 to 9. Alternatively, the filovirus peptide comprising one or more of the CD8+ T cell epitopes set out in SEQ ID NOs: 1 to 9 may comprise two or more, such as three or more, four or more, five or more, six or more, seven or more, or eight or more of the CD8+ T cell epitopes set out in SEQ ID NOs: 1 to 9, in any combination. The filovirus peptide comprising one or more of the CD8+ T cell epitopes set out in SEQ ID NOs: 1 to 9 may comprise all of the CD8+ T cell epitopes set out in SEQ ID NOs: 1 to 9.

As well as one or more of the CD8+ T cell epitopes set out in SEQ ID NOs: 1 to 9, the filovirus peptide may comprise one or more other CD8+ T cell epitopes, one or more CD4+ T cell epitopes and/or one or more B cell epitopes. For example, the filovirus peptide may comprise two or more, such as three or more, four or more, five or more, ten or more, fifteen or more, or twenty or more CD8+ T cell epitopes other than those set out in SEQ ID NOs: 1 to 9. The filovirus peptide may comprise two or more, such as three or more, four or more, five or more, ten or more, fifteen or more, or twenty or more CD4+ T cell epitopes. The filovirus peptide may comprise two or more, such as three or more, four or more, five or more, ten or more, fifteen or more, or twenty or more B cell epitopes.

The vaccine composition may comprise two or more filovirus peptides each comprising a CD8+ T cell epitope comprising a different sequence selected from SEQ ID NOs: 1 to 9. Each of the filovirus peptides may be have any of the properties set out in the preceding paragraphs. For instance, each filovirus peptide may comprise multiple CD8+ T cell epitopes set out in SEQ ID NOs: 1 to 9 and, optionally, one or more other CD8+ T cell epitopes, one or more CD4+ T cell epitopes and/or one or more B cell epitopes. In one aspect, the vaccine composition may comprise three or more, four or more, five or more, six or more, seven or more, or eight or more filovirus peptides each comprising a CD8+ T cell epitope comprising a different sequence selected from SEQ ID NOs: 1 to 9. The vaccine composition may, for example, comprise 9 filovirus peptides each comprising a CD8+ T cell epitope comprising a different sequence selected from SEQ ID NOs: 1 to 9.

The vaccine composition may further comprise one or more (such as about 1 to 50, 2 to 40, 3 to 30, 4 to 25, 5 to 20, 6 to 15, 7, 8, 9 or 10) additional peptides each comprising one or more epitopes. The epitope may be a CD8+ T cell epitope, a CD4+ T cell epitope and/or a B cell epitope. The CD8+ T cell epitope is preferably a CD8+ T cell epitope other than the CD8+ T cell epitopes set out in SEQ ID NOs: 1 to 9. The CD8+ T cell epitope may, for example, be a filovirus CD8+ epitope, i.e. a peptide that is expressed by one or more filoviruses and that is that is capable of (i) presentation by a class I MHC molecule and (ii) recognition by a T cell receptor (TCR) present on a CD8+ T cell. Alternatively, the CD8+ T cell epitope may be an CD8+ T cell epitope that is not expressed by one or more filoviruses. The CD4+ T cell epitope may, for example, be a filovirus CD4+ epitope, i.e. a peptide that is expressed by one or more filoviruses and that is that is capable of (i) presentation by a class II MHC molecule and (ii) recognition by a T cell receptor (TCR) present on a CD4+ T cell. Alternatively, the CD4+ T cell epitope may be an CD4+ T cell epitope that is not expressed by one or more filoviruses. CD8+ and CD4+ T cell epitopes are described in more detail below.

A filovirus peptide is a peptide that is expressed by one or more filoviruses. Numerous species of filovirus exist across two main genera, ebolaviruses and marburgviruses. Ebolavirus species include Zaire ebolavirus (ZEBOV), Bundibugyo ebolavirus (BDBV), Reston ebolavirus (RESTV), Sudan ebolavirus (SUDV), and Taï Forest ebolavirus (TAFV). Marburgvirus species include Marburg virus (MARV) and Ravn virus (RAVV). A third genus, cuevaviruses, includes the Lloviu virus species.

Any filovirus peptide comprised in the vaccine composition of the invention may comprise a peptide that is expressed by one or more of Zaire ebolavirus, Bundibugyo ebolavirus, Reston ebolavirus, Sudan ebolavirus, Taï Forest ebolavirus, Marburg virus, Ravn virus and Lloviu virus. For instance, the filovirus peptide comprising one or more of the CD8+ T cell epitopes set out in SEQ ID NOs: 1 to 9 may be expressed by Zaire ebolavirus, Bundibugyo ebolavirus, Reston ebolavirus, Sudan ebolavirus, Taï Forest ebolavirus, Marburg virus, Ravn virus and/or Lloviu virus. Likewise, when the composition comprises an additional peptide that is a filovirus peptide, that additional filovirus peptide may be expressed by Zaire ebolavirus, Bundibugyo ebolavirus, Reston ebolavirus, Sudan ebolavirus, Taï Forest ebolavirus, Marburg virus, Ravn virus and/or Lloviu virus. Accordingly, the vaccine composition may comprise filovirus peptides from one or more species of filovirus, such as 1 to 8, 2 to 7, 3 to 6, or 4 to 5 species of filovirus.

The filovirus peptide may be a peptide that is expressed on the surface of one or more filoviruses, or intracellularly within one or more filoviruses. The peptide may be a structural peptide or a functional peptide, such as a peptide involved in the metabolism or replication of the filovirus. Preferably, the peptide is an internal peptide. Preferably, the peptide is conserved between two or more different filovirus strains.

The filovirus peptide may contain any number of amino acids, i.e. be of any length. Typically, the filovirus peptide is about 8 to about 30, 35 or 40 amino acids in length, such as about 9 to about 29, about 10 to about 28, about 11 to about 27, about 12 to about 26, about 13 to about 25, about 13 to about 24, about 14 to about 23, about 15 to about 22, about 16 to about 21, about 17 to about 20, or about 18 to about 29 amino acids in length.

The filovirus peptide may be chemically derived from a polypeptide filovirus antigen, for example by proteolytic cleavage. More typically, the filovirus peptide may be synthesised using methods well known in the art.

The term “peptide” includes not only molecules in which amino acid residues are joined by peptide (—CO—NH—) linkages but also molecules in which the peptide bond is reversed. Such retro-inverso peptidomimetics may be made using methods known in the art, for example such as those described in Meziere et al (1997) J. Immunol. 159, 3230-3237. This approach involves making pseudopeptides containing changes involving the backbone, and not the orientation of side chains. Meziere et al (1997) show that, at least for MHC class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond. It will also be appreciated that the peptide may conveniently be blocked at its N- or C-terminus so as to help reduce susceptibility to exoproteolytic digestion. For example, the N-terminal amino group of the peptides may be protected by reacting with a carboxylic acid and the C-terminal carboxyl group of the peptide may be protected by reacting with an amine. Other examples of modifications include glycosylation and phosphorylation. Another potential modification is that hydrogens on the side chain amines of R or K may be replaced with methylene groups (—NH2 may be modified to —NH(Me) or —N(Me)₂).

The term “peptide” also includes peptide variants that increase or decrease the half-life of the peptide in vivo. Examples of analogues capable of increasing the half-life of peptides used according to the invention include peptoid analogues of the peptides, D-amino acid derivatives of the peptides, and peptide-peptoid hybrids. A further embodiment of the variant polypeptides used according to the invention comprises D-amino acid forms of the polypeptide. The preparation of polypeptides using D-amino acids rather than L-amino acids greatly decreases any unwanted breakdown of such an agent by normal metabolic processes, decreasing the amounts of agent which needs to be administered, along with the frequency of its administration.

### CD8+ T Cell Epitopes

The vaccine composition of the invention comprises a filovirus peptide comprising one or more of the CD8+ T cell epitopes set out in SEQ ID NOs: 1 to 9 (see Table 1). The filovirus peptide comprising one or more of the CD8+ T cell epitopes set out in SEQ ID NOs: 1 to 9 may further comprise one or more (such as two or more, three or more, four or more, five or more, ten or more, fifteen or more, or twenty or more) other CD8+ T cell epitopes. The vaccine composition may further comprise one or more (such as 1 to 50, 2 to 40, 3 to 30, 4 to 25, 5 to 20, 6 to 15, 7, 8, 9 or 10) additional peptides each comprising one or more CD8+ T cell epitopes. Preferably, the additional peptide is a filovirus peptide.

A CD8+ T cell epitope is a peptide that is capable of (i) presentation by a class I MHC molecule and (ii) recognition by a T cell receptor (TCR) present on a CD8+ T cell. Preferably, recognition by the TCR results in activation of the CD8+ T cell. CD8+ T cell activation may lead to increased proliferation, cytokine production and/or cyotoxic effects.

Typically, the CD8+ T cell epitope is around 9 amino acids in length. The CD8+ T cell epitope may though be shorter or longer. For example, the CD8+ T cell epitope may be about 8, 9, 10, 11, 12, 13, 14 or 15 amino acids in length. The CD8+ T cell epitope may be about 8 to 15, 9 to 14 or 10 to 12 amino acids in length.

Filovirus peptides comprising a CD8+ T cell epitope are known in the art. Methods for identifying CD8+ T cell epitopes are known in the art. Epitope mapping methods include X-ray co-crystallography, array-based oligo-peptide scanning (sometimes called overlapping peptide scan or pepscan analysis), site-directed mutagenesis, high throughput mutagenesis mapping, hydrogen-deuterium exchange, crosslinking coupled mass spectrometry, phage display and limited proteolysis. MHC motif prediction methodologies may also be used.

CD8+ T cell epitopes presented by filovirus-infected cells can be identified in order to directly identify CD8+ T cell epitopes for inclusion in the vaccine composition. This is an efficient and logical method which can be used alone or to confirm the utility of potential CD8+ T cell epitopes identified in silico from the filovirus (e.g. EBOV) genome, for example by MHC motif prediction methodologies.

To perform the method, cells are infected with a filovirus and maintained in culture for a period of around 72 hours at a temperature of around 37° C. Following culture, the cells are then harvested and washed. Next, the cells are lysed, for instance by homogenisation and freezing/thawing in buffer containing 1% NP40. Lysates are cleared by centrifugation at 2000 rpm for 30 minutes to remove cell debris.

MHC/peptide complexes are then isolated from the lysates by immunoaffinity chromatography using protein A/G beads (UltraLink Immobilized Protein A/G, pierce, Rockford, Ill.) coated with W6/32 (a monoclonal antibody recognising pan MHC class I molecule). To coat the beads with the antibody, the beads are washed with low pH buffer followed by PBS rinses, incubated with 0.5 mg of the antibody at room temperature for 2 hours, and washed three times to remove unbound antibody. For immunoaffinity chromatography, the coated beads are incubate with lysate for 2 hours at room temperature with continuous rocking. The beads are then separated from the lysate by centrifuging at 1000 rpm for 5 minutes. Bound MHC complexes are eluted from the beads by the addition of 0.1% trifluoroacetic acid (TFA), pH 1.5.

The eluate is next heated at 85° C. for 15 minutes to dissociate the bound peptides from the MHC molecules. After cooling to room temperature, peptides are separated from the antibody by centrifugation using, for example, 3 kDa molecular mass cutoff membrane filters (Millipore). The filtrate is concentrated using vacuum centrifugation and reconstituted to a final volume of 1004 The purified peptide mixture is fractionated, for example using a C-18 reversed phase (RP) column (e.g. 4.6 mm diameter×150 mm length) using an offline HPLC. For this step, mobile phase A may be 2% acetonitrile (CAN) and 0.1% formic acid (FA) in water, while mobile phase B may be 0.1% FA and 90% CAN in water.

The peptide-containing fractions are then eluted from the column, dried under a vacuum, and analysed by mass spectrometry to identify the sequences of the fractions. The acquired spectral data can then be searched against all databased filovirus proteins to identify peptide sequences associated with filovirus. Synthetic peptides may then be made according to the identified sequences and subjected to mass spectrometry to confirm their identity to the peptides in the peptide-containing fractions.

In this method, any type of cells may be infected with filovirus. The cells may be antigen presenting cells. The cells may be hepatoma cells such as HepG2 cells, EBV-transformed lymphoblastoid B cells such as JY cells, or lymphoblasts such as T2 cells.

Likewise, any filovirus of interest may be used to infect the cells. For instance, the filovirus may be an ebolavirus or a marburgvirus. The ebolavirus may, for example, be Zaire ebolavirus, Bundibugyo ebolavirus, Reston ebolavirus, Sudan ebolavirus or Taï Forest ebolavirus. The marburgbirus may, for example, be Marburg virus or Ravn virus.

The direct identification of CD8+ T cell epitopes presented by filovirus-infected cells is advantageous compared to MHC motif prediction methodologies. The immune epitope database (IEDB; http://www.iedb.org) is generated by motif prediction methods, and not functional methods, and contains numerous predicted HLA-specific filovirus T cell epitopes, including some shared epitopes with high MHC binding scores and limited CTL characterization. As both dominant and subdominant epitopes may be presented by filovirus-infected cells, it is difficult to sort out the dominance hierarchies of naturally presented epitopes using the database. Thus, it is not clear from the immune epitope database alone which of the listed epitopes may be expected to efficiently induce a CD8+ T cell response when included in a vaccine composition. The direct identification method set out above provides a mechanism for confirming the utility of the epitopes.

In the case that CD8+ T cell epitopes are identified in silico from the filovirus (e.g. EBOV) genome, for example by MHC motif prediction methodologies, in vitro or in vivo assays may be performed to confirm that the identified epitope does indeed function as a CD8+ T cell epitope. For example, MHC class I binding assays may be performed. The ability of the epitope to induce a cytotoxic T lymphocyte (CTL) response may be evaluated. Suitable assays are known in the art and are described, for example, in WO 2012/050193. For instance, Example 1 below describes the verification of certain CD8+ T cell epitopes identified in silico using by investigating the ability of the epitopes to elicit CTL responses in vitro.

Vaccine compositions based on epitopes presented by filovirus-infected cells, such as the vaccine composition of the invention, are superior to vaccines based on a viral glycoprotein or protein subunit or a motif predicted epitope. Protein processing by the immune system is likely to alter native viral epitopes. Basing a vaccine composition on peptides demonstrated to be presented by infected cells and/or to induce an anti-filovirus CTL response removes this source of uncertainty, because the peptides have already undergone protein processing.

As set out in the Examples, the present inventors confirmed that the known peptides KIIKFLEPL (P1; SEQ ID NO: 1), KYTMQDALF (SEQ ID NO: 6), QYADCELHL (SEQ ID NO: 8) and SLTDRELLL (P6; SEQ ID NO: 9) are CD8+ T cell epitopes conserved between ebolaviruses and marburgviruses. Thus, these CD8+ T cell epitopes are suitable for inclusion in a cross-protective vaccine. The method described above for the direct identification of CD8+ T cell epitopes may be used to identify conserved CD8+ T cell epitopes presented by cells infected by different filoviruses, in order to identify CD8+ T cell epitopes suitable for inclusion in a cross-protective vaccine.

### Cross Protective Vaccine Compositions

As shown in Table 1, many of the CD8+ T cell epitopes set out in SEQ ID NOs: 1 to 9 are conserved CD8+ T cell epitopes that are presented by MHC molecules on cells infected by different filoviruses. Accordingly, an immune response generated by vaccination with a composition that comprises any of these epitopes should protect against subsequent infection with any filovirus that shares that epitope. In other words, the vaccine composition of the invention may have built-in cross-species and/or cross-genus efficacy, i.e. be a cross-protective filovirus vaccine composition.

The inclusion of a filovirus peptide comprising a conserved CD8+ T cell epitope in the vaccine composition of the invention is therefore advantageous. The cross-protective capabilities of such a composition may permit swift ring vaccination in the case of an outbreak of haemorrhagic fever without the need to first determine the particular virus involved in the disease; allow use of the vaccine at the population level to induce widespread immunity to filoviruses, guarding against future outbreaks; provide a cost-effective option for filovirus vaccination in the developing countries where outbreaks of filovirus haemorrhagic fevers most commonly occur; and protect against emerging filovirus strains.

Interaction with HLA Supertypes

The vaccine composition may comprise at least two filovirus peptides comprising a CD8+ T cell epitope which each interacts with a different HLA supertype. Including a plurality of such peptides in the vaccine composition allows the vaccine composition to elicit a CD8+ T cell response in a greater proportion of individuals to which the vaccine composition is administered. This is because the vaccine composition should be capable of eliciting a CD8+ T cell response in all individuals of an HLA supertype that interacts with one of the CD8+ T cell epitopes comprised in the plurality of filovirus peptides. Each CD8+ T cell epitope may interact with HLA-A1, HLA-A2, HLA-A3, HLA-A24, HLA-B7, HLA-B8, HLA-B27, HLA-B44, HLA-B58 or HLA-B62, or any other HLA supertype know in the art. Any combination of filovirus peptides comprising such a CD8+ T cell epitope is possible.

The vaccine composition may comprise at least one filovirus peptide comprising a CD8+ T cell epitope which interacts at least two different HLA supertypes. Again, this allows the vaccine composition to elicit a CD8+ T cell response in a greater proportion of individuals to which the vaccine composition is administered. The vaccine composition may comprise at least two, at least three, at least four, at least five, at least two, at least fifteen, or at least twenty filovirus peptides comprising a CD8+ T cell epitope that each interact with at least two different HLA subtypes. Each filovirus peptide may interact with at least two, at least three, at least four, at least five, at least six, at least 7, at least 8, at least 9 or at least 10 different HLA supertypes. Each filovirus peptide may interact with two or more of HLA-A1, HLA-A2, HLA-A3, HLA-A24, HLA-B7, HLA-B8, HLA-B27, HLA-B44, HLA-B58 or HLA-B62, or any other HLA supertype known in the art, in any combination. Preferably, the vaccine composition comprises a filovirus peptide comprising a CD8+ T cell epitope that interacts with HLA-A2 and HLA-24. In this case, the vaccine composition may, for example, comprise a filovirus peptide comprising a CD8+ T cell set out in SEQ ID NO: 1 or SEQ ID NO: 9.

### CD4+ T Cell Epitopes

The vaccine composition of the invention may comprise a peptide comprising a CD4+ T cell epitope. The vaccine composition may comprise two or more, such as three or more, four or more, five our more, ten or more, fifteen or more or twenty or more peptides comprising a CD4+ T cell epitope. A CD4+ T cell epitope is a peptide that is capable of (i) presentation by a class II MHC molecule and (ii) recognition by a T cell receptor (TCR) present on a CD4+ T cell. Preferably, recognition by the TCR results in activation of the CD4+ T cell. CD4+ T cell activation may lead to increased proliferation and/or cytokine production.

The CD4+ T cell epitope may be a filovirus CD4+ T cell epitope. That is, the CD4+ T cell epitope may be a peptide that is expressed by one or more filoviruses and that is that is capable of (i) presentation by a class II MHC molecule and (ii) recognition by a T cell receptor (TCR) present on a CD4+ T cell. Such peptides are known in the art.

The CD4+ T cell epitope may be a CD4+ T cell epitope other than a filovirus CD4+ T cell epitope. For example, the CD4+ T cell may be expressed by an organism other than a filovirus. The CD4+ T cell epitope may, for example, be expressed by Clostriudium tetani. For instance, the CD4+ T cell epitope may be derived from tetanus toxin.

The CD4+ T cell epitope may be a CD4+ T cell epitope that reacts with all class II HLA types, i.e. a so-called “promiscuous” epitope. Inclusion of a promiscuous epitope in the vaccine composition may improve the ability of the vaccine composition to induce an immune response to the filovirus peptide comprising one or more of the CD8+ T cell epitopes set out in SEQ ID NOs: 1 to 9. The CD4+ T cell epitope may, for example, comprise the sequence FKLQTMVKLFNRIKNNVA (SEQ ID NO: 10) and/or the sequence LQTMVKLFNRIKNNVAGGC (SEQ ID NO: 11). SEQ ID NOs 10 and 11 are promiscuous epitopes derived from tetanus toxin.

The peptide comprising a CD4+ T cell epitope may be a different peptide from the filovirus peptide comprising one or more of the CD8+ T cell epitopes set out in SEQ ID NOs: 1 to 9. The CD4+ T cell epitope may, for instance, be comprised in an additional peptide in the vaccine composition, i.e. in a peptide that does not comprise one or more of the CD8+ T cell epitopes set out in SEQ ID NOs: 1 to 9. As mentioned above, the additional peptide may comprise one or more CD8+ T cell epitopes and/or one or more B cell epitopes as well as the CD4+ T cell epitope. For instance, the additional peptide may comprise one or more filovirus CD8+ T cell epitopes.

The peptide comprising a CD4+ T cell epitope may be the same peptide as the filovirus peptide comprising one or more of the CD8+ T cell epitopes set out in SEQ ID NOs: 1 to 9. That is, the filovirus peptide comprising one or more of the CD8+ T cell epitopes set out in SEQ ID NOs: 1 to 9 may further comprise a CD4+ T cell epitope.

When the peptide comprising a CD4+ T cell epitope also comprises a CD8+ T cell epitope (such as one or more of the CD8+ T cell epitopes set out in SEQ ID NOs: 1 to 9), the CD8+ epitope may be nested within the CD4+ T cell epitope. CD4+ T cell epitopes are typically longer than CD8+ T cell epitopes. Therefore, extending one or both termini of the CD8+ T cell epitope may yield a longer, CD4+ T cell epitope whose sequence still comprises the CD8+ T cell epitope. Therefore, the CD4+ T cell epitope may comprise a CD8+ T cell epitope, such as a CD8+ T cell epitope set out in SEQ ID NOs: 1 to 9, extended at its N-terminus or C-terminus. The CD8+ T cell epitope may be extended by 1, 2, 3, 4 or 5 amino acids at its N terminus. The CD8+ T cell epitope may be extended by 1, 2, 3, 4 or 5 amino acids at its C terminus. Preferably, the CD8+ T cell epitope is extended by 3 amino acids at the N terminus, and 3 amino acids at the C terminus. However, the CD8+ T cell epitope need not be extended by the same number of amino acids at each terminus.

The CD8+ T cell epitope nested within a CD4+ T cell epitope may be capable of generating a robust CTL response. The extended peptide (CD4+ T cell epitope) may be capable of inducing T helper mediated cytokine responses. Thus, inclusion of a filovirus peptide comprising a CD8+ T cell epitope and a CD4+ T cell epitope in the vaccine composition may allow the vaccine composition to induce both cytotoxic and helper T cell responses.

The filovirus peptide comprising a CD4+ T cell epitope may be attached to a nanoparticle. When the peptide comprising a CD4+ T cell epitope is a different peptide from the filovirus peptide comprising one or more of the CD8+ T cell epitopes set out in SEQ ID NOs: 1 to 9, each peptide may be attached to the same nanoparticle or to a different nanoparticle. The nanoparticle may be a gold nanoparticle. Nanoparticles and attachment thereto are described below.

### B Cell Epitopes

The vaccine composition of the invention may comprise a peptide comprising a B cell epitope. The vaccine composition may comprise two or more, such as three or more, four or more, five our more, ten or more, fifteen or more or twenty or more peptides comprising a B cell epitope. A B cell epitope is a peptide that is capable of recognition by a B cell receptor (BCR) present on a B cell. Preferably, recognition by the BCR results in activation and/or maturation of the B cell. B cell activation may lead to increased proliferation, and/or antibody production.

The B cell epitope may be a filovirus CD4+ T cell epitope. That is, the B cell epitope may be a peptide that is expressed by one or more filoviruses and that is capable of recognition by a B cell receptor (BCR) present on a B cell. Such peptides are known in the art.

The B cell epitope may be a linear epitope, i.e. an epitope that is defined by the primary amino acid sequence of a particular region of a filovirus protein. Alternatively, the epitope may be a conformational epitope, i.e. an epitope that is defined by the conformational structure of a native filovirus protein. In this case, the epitope may be continuous (i.e. the components that interact with the antibody are situated next to each other sequentially on the protein) or discontinuous (i.e. the components that interact with the antibody are situated on disparate parts of the protein, which are brought close to each other in the folded native protein structure).

Typically, the B cell epitope is around 5 to 20 amino acids in length, such as 6 to 19, 7 to 18, 8 to 17, 9 to 16, 10 to 15, 11 to 14 or 12 to 13 amino acids in length.

Methods for identifying B cell epitopes are also known in the art. For instance, epitope mapping methods may be used to identify B cell epitopes. These methods include structural approaches, wherein the known or modelled structure of a protein is be used in an algorithm based approach to predict surface epitopes, and functional approaches, wherein the binding of whole proteins, protein fragments or peptides to an antibody can be quantitated e.g. using an Enzyme-Linked Immunosorbent Assay (ELISA). Competition mapping, antigen modification or protein fragmentation methods may also be used.

### Nanoparticles

In the vaccine composition of the invention, the filovirus peptide comprising one or more of the CD8+ T cell epitopes set out in SEQ ID NOs: 1 to 9 is attached to a nanoparticle. Any other peptides further comprised in the vaccine composition may also be attached to a nanoparticle. Attachment to a nanoparticle, for example a gold nanoparticle, is beneficial.

As set out above and demonstrated in the Examples below, attachment of the peptide to a nanoparticle (such as a gold nanoparticle) reduces or eliminates the need to include a virus or an adjuvant in the vaccine composition. The nanoparticles may contain immune “danger signals” that help to effectively induce an immune response to the peptides. The nanoparticles may induce dendritic cell (DC) activation and maturation, required for a robust immune response. The nanoparticles may contain non-self components that improve uptake of the nanoparticles and thus the peptides by cells, such as antigen presenting cells. Attachment of a peptide to a nanoparticle may therefore enhance the ability of antigen presenting cells to stimulate virus-specific T and/or B cells. Attachment to a nanoparticle also facilitates delivery of the vaccine compositions via the subcutaneous, intradermal, transdermal and oral/buccal routes, providing flexibility in administration.

Nanoparticles are particles between 1 and 100 nanometers (nm) in size which can be used as a substrate for immobilising ligands. In the vaccine compositions of the invention, the nanoparticle may have a mean diameter of 1 to 100, 20 to 90, 30 to 80, 40 to 70 or 50 to 60 nm. Preferably, the nanoparticle has a mean diameter of 20 to 40 nm. A mean diameter of 20 to 40 nm facilitates uptake of the nanoparticle to the cytosol. The mean diameter can be measured using techniques well known in the art such as transmission electron microscopy.

Nanoparticles suitable for the delivery of antigen, such as a filovirus peptide, are known in the art. Methods for the production of such nanoparticles are also known.

The nanoparticle may, for example, be a polymeric nanoparticle, an inorganic nanoparticle, a liposome, an immune stimulating complex (ISCOM), a virus-like particle (VLP), or a self-assembling protein. The nanoparticle is preferably a calcium phosphate nanoparticle, a silicon nanoparticle or a gold nanoparticle.

The nanoparticle may be a polymeric nanoparticle. The polymeric nanoparticle may comprise one or more synthetic polymers, such as poly(d,l-lactide-co-glycolide) (PLG), poly(d,l-lactic-coglycolic acid) (PLGA), poly(g-glutamic acid) (g-PGA)m poly(ethylene glycol) (PEG), or polystyrene. The polymeric nanoparticle may comprise one or more natural polymers such as a polysaccharide, for example pullulan, alginate, inulin, and chitosan. The use of a polymeric nanoparticle may be advantageous due to the properties of the polymers that may be include in the nanoparticle. For instance, the natural and synthetic polymers recited above may have good biocompatibility and biodegradability, a non-toxic nature and/or the ability to be manipulated into desired shapes and sizes. The polymeric nanoparticle may form a hydrogel nanoparticle. Hydrogel nanoparticles are a type of nano-sized hydrophilic three-dimensional polymer network. Hydrogel nanoparticles have favourable properties including flexible mesh size, large surface area for multivalent conjugation, high water content, and high loading capacity for antigens. Polymers such as Poly(L-lactic acid) (PLA), PLGA, PEG, and polysaccharides are particularly suitable for forming hydrogel nanoparticles.

The nanoparticle may be an inorganic nanoparticle. Typically, inorganic nanoparticles have a rigid structure and are non-biodegradable. However, the inorganic nanoparticle may be biodegradable. The inorganic nanoparticle may comprise a shell in which an antigen may be encapsulated. The inorganic nanoparticle may comprise a core to which an antigen may be covalently attached. The core may comprise a metal. For example, the core may comprise gold (Au), silver (Ag) or copper (Cu) atoms. The core may be formed of more than one type of atom. For instance, the core may comprise an alloy, such as an alloy of Au/Ag, Au/Cu, Au/Ag/Cu, Au/Pt, Au/Pd or Au/Ag/Cu/Pd. The core may comprise calcium phosphate (CaP). The core may comprise a semiconductor material, for example cadmium selenide.

Other exemplary inorganic nanoparticles include carbon nanoparticles and silica-based nanoparticles. Carbon nanoparticles are have good biocompatibility and can be synthesized into nanotubes and mesoporous spheres. Silica-based nanoparticles (SiNPs) are biocompatible and can be prepared with tunable structural parameters to suit their therapeutic application.

The nanoparticle may be a silicon nanoparticle, such as an elemental silicon nanoparticle. The nanoparticle may be mesoporous or have a honeycomb pore structure. Preferably, the nanoparticle is an elemental silicon particle having a honeycomb pore structure. Such nanoparticles are known in the art and offer tunable and controlled drug loading, targeting and release that can be tailored to almost any load, route of administration, target or release profile. For example, such nanoparticles may increase the bioavailability of their load, and/or improve the intestinal permeability and absorption of orally administered actives. The nanoparticles may have an exceptionally high loading capacity due to their porous structure and large surface area. The nanoparticles may release their load over days, weeks or months, depending on their physical properties. Since silicon is a naturally occurring element of the human body, the nanoparticles may elicit no response from the immune system. This is advantageous to the in vivo safety of the nanoparticles.

Any of the SiNPs described above may be biodegradable or non-biodegradable. A biodegradable SiNP may dissolve to orthosilic acid, the bioavailable form of silicon. Orthosilic acid has been shown to be beneficial for the health of bones, connective tissue, hair, and skin.

The nanoparticle may be a liposome. Liposomes are typically formed from biodegradable, non-toxic phospholipids and comprise a self-assembling phospholipid bilayer shell with an aqueous core. A liposome may be an unilameller vesicle comprising a single phospholipid bilayer, or a multilameller vesicle that comprises several concentric phospholipid shells separated by layers of water. As a consequence, liposomes can be tailored to incorporate either hydrophilic molecules into the aqueous core or hydrophobic molecules within the phospholipid bilayers. Liposomes may encapsulate antigen within the core for delivery. Liposomes may incorporate viral envelope glycoproteins to the shell to form virosomes. A number of liposome-based products are established in the art and are approved for human use.

The nanoparticle may be an immune-stimulating complex (ISCOM). ISCOMs are cage-like particles which are typically formed from colloidal saponin-containing micelles. ISCOMs may comprise cholesterol, phospholipid (such as phosphatidylethanolamine or phosphatidylcholine) and saponin (such as Quil A from the tree Quillaia saponaria). ISCOMs have traditionally been used to entrap viral envelope proteins, such as envelope proteins from herpes simplex virus type 1, hepatitis B, or influenza virus.

The nanoparticle may be a virus-like particle (VLP). VLPs are self-assembling nanoparticles that lack infectious nucleic acid, which are formed by self-assembly of biocompatible capsid protein. VLPs are typically about 20 to about 150 nm, such as about 20 to about 40 nm, about 30 to about 140 nm, about 40 to about 130 nm, about 50 to about 120 nm, about 60 to about 110 nm, about 70 to about 100 nm, or about 80 to about 90 nm in diameter. VLPs advantageously harness the power of evolved viral structure, which is naturally optimized for interaction with the immune system. The naturally-optimized nanoparticle size and repetitive structural order means that VLPs induce potent immune responses, even in the absence of adjuvant.

The nanoparticle may be a self-assembling protein. For instance, the nanoparticle may comprise ferritin. Ferritin is a protein that can self-assemble into nearly-spherical 10 nm structures. The nanoparticle may comprise major vault protein (MVP). Ninety-six units of MVP can self-assemble into a barrel-shaped vault nanoparticle, with a size of approximately 40 nm wide and 70 nm long.

The nanoparticle may be a calcium phosphate (CaP) nanoparticle. CaP nanoparticles may comprise a core comprising one or more (such as two or more, 10 or more, 20 or more, 50 or more, 100 or more, 200 or more, or 500 or more) molecules of CaP. CaP nanoparticles and methods for their production are known in the art. For instance, a stable nano-suspension of CAP nanoparticles may be generated by mixing inorganic salt solutions of calcium and phosphates in pre-determined ratios under constant mixing.

The CaP nanoparticle may have an average particle size of about 80 to about 100 nm, such as about 82 to about 98 nm, about 84 to about 96 nm, about 86 to about 94 nm, or about 88 to about 92 nm. This particle size may produce a better performance in terms of immune cell uptake and immune response than other, larger particle sizes. The particle size may be stable (i.e. show no significant change), for instance when measured over a period of 1 month, 2 months, 3 months, 6 months, 12 months, 18 months, 24 months, 36 months or 48 months.

CaP nanoparticles can be co-formulated with one or multiple antigens either adsorbed on the surface of the nanoparticle or co-precipitated with CaP during particle synthesis. For example, a peptide, such as a filovirus peptide, may be attached to the CaP nanoparticle by dissolving the peptide in DMSO (for example at a concentration of about 10 mg/ml), adding to a suspension of CaP nanoparticles together with N-acetyl-glucosamine (GlcNAc) (for example at 0.093 mol/L and ultra-pure water, and mixing at room temperature for a period of about 4 hours (for example, 1 hour, 2 hours, 3 hours, 5 hours, 6 hours, 7 hours, 8 hours, 9 hours or 10 hours).

The vaccine composition may comprise about 0.15 to about 0.8%, such as 0.2 to about 0.75%, 0.25 to about 0.7%, 0.3 to about 0.6%, 0.35 to about 0.65%, 0.4 to about 0.6%, or 0.45 to about 0.55%, CaP nanoparticles. Preferably the vaccine composition comprises about 0.3% CaP nanoparticles.

CaP nanoparticles have a high degree of biocompatibility due to their chemical similarity to human hard tissues such as bone and teeth. Advantageously, therefore, CaP nanoparticles are non-toxic when used for therapeutic applications. CaP nanoparticles are safe for administration via intramuscular, subcutaneous, oral, or inhalation routes. CaP nanoparticles are also simple to synthesise commercially. Furthermore, CaP nanoparticles may be associated with slow release of antigen, which may enhance the induction of an immune response to a peptide attached to the nanoparticle. CaP nanoparticles may be used both as an adjuvant, and as a drug delivery vehicle.

The nanoparticle may be a gold nanoparticle. Gold nanoparticles are known in the art and are described in particular in WO 2002/32404, WO 2006/037979, WO 2007/122388, WO 2007/015105 and WO 2013/034726. The gold nanoparticle attached to each peptide may be a gold nanoparticle described in any of WO 2002/32404, WO 2006/037979, WO 2007/122388, WO 2007/015105 and WO 2013/034726.

Gold nanoparticles comprise a core comprising a gold (Au) atom. The core may further comprise one or more Fe, Cu or Gd atoms. The core may be formed from a gold alloy, such as Au/Fe, Au/Cu, Au/Gd, Au/Fe/Cu, Au/Fe/Gd or Au/Fe/Cu/Gd. The total number of atoms in the core may be 100 to 500 atoms, such as 150 to 450, 200 to 400 or 250 to 350 atoms. The gold nanoparticle may have a mean diameter of 1 to 100, 20 to 90, 30 to 80, 40 to 70 or 50 to 60 nm. Preferably, the gold nanoparticle has a mean diameter of 20 to 40 nm.

The nanoparticle may comprise a surface coated with alpha-galactose and/or beta-GlcNHAc. For instance, the nanoparticle may comprise a surface passivated with alpha-galactose and/or beta-GlcNHAc. In this case, the nanoparticle may, for example, be a nanoparticle which comprises a core including metal and/or semiconductor atoms. For instance, the nanoparticle may be a gold nanoparticle. Beta-GlcNHAc is a bacterial pathogen-associated-molecular pattern (PAMP), which is capable of activating antigen-presenting cells. In this way, a nanoparticle comprising a surface coated or passivated with Beta-GlcNHAc may non-specifically stimulate an immune response. Attachment of the filovirus peptide comprising one or more of the CD8+ T cell epitopes set out in SEQ ID NOs: 1 to 9 to such a nanoparticle may therefore improve the immune response elicited by administration of the vaccine composition of the invention to an individual.

One or more ligands other than the peptide may be linked to the nanoparticle, which may be any of the types of nanoparticle described above. The ligands may form a “corona”, a layer or coating which may partially or completely cover the surface of the core. The corona may be considered to be an organic layer that surrounds or partially surrounds the nanoparticle core. The corona may provide or participate in passivating the core of the nanoparticle. Thus, in certain cases the corona may be a sufficiently complete coating layer to stabilise the core. The corona may facilitate solubility, such as water solubility, of the nanoparticles of the present invention.

The nanoparticle may comprise at least 10, at least 20, at least 30, at least 40 or at least 50 ligands. The ligands may include one or more peptides, protein domains, nucleic acid molecules, lipidic groups, carbohydrate groups, anionic groups, or cationic groups, glycolipids and/or glycoproteins. The carbohydrate group may be a polysaccharide, an oligosaccharide or a monosaccharide group (e.g. glucose). One or more of the ligands may be a non-self component, that renders the nanoparticle more likely to be taken up by antigen presenting cells due to its similarity to a pathogenic component. For instance, one or more ligands may comprise a carbohydrate moiety (such as a bacterial carbohydrate moiety), a surfactant moiety and/or a glutathione moiety. Exemplary ligands include glucose, N-acetylglucosamine (GlcNAc), glutathione, 2′-thioethyl-β-D-glucopyranoside and 2′-thioethyl-D-glucopyranoside. Preferred ligands include glycoconjugates, which form glyconanoparticles

Linkage of the ligands to the core may be facilitated by a linker. The linker may comprise a thiol group, an alkyl group, a glycol group or a peptide group. For instance, the linker may comprise C2-C15 alkyl and/or C2-C15 glycol. The linker may comprise a sulphur-containing group, amino-containing group, phosphate-containing group or oxygen-containing group that is capable of covalent attachment to the core. Alternatively, the ligands may be directly linked to the core, for example via a sulphur-containing group, amino-containing group, phosphate-containing group or oxygen-containing group comprised in the ligand.

### Attachment to Nanoparticles

The peptide may be attached at its N-terminus to the nanoparticle. Typically, the peptide is attached to the core of the nanoparticle, but attachment to the corona or a ligand may also be possible.

The peptide may be directly attached to the nanoparticle, for example by covalent bonding of an atom in a sulphur-containing group, amino-containing group, phosphate-containing group or oxygen-containing group in the peptide to an atom in the nanoparticle or its core.

A linker may be used to link the peptide to the nanoparticle. The linker may comprise a sulphur-containing group, amino-containing group, phosphate-containing group or oxygen-containing group that is capable of covalent attachment to an atom in the core. For example, the linker may comprise a thiol group, an alkyl group, a glycol group or a peptide group.

The linker may comprise a peptide portion and a non-peptide portion. The peptide portion may comprise the sequence X₁X₂Z₁, wherein X₁ is an amino acid selected from A and G; X₂ is an amino acid selected from A and G; and Z₁ is an amino acid selected from Y and F. The peptide portion may comprise the sequence AAY or FLAAY. The peptide portion of the linker may be linked to the N-terminus of the peptide. The non-peptide portion of the linker may comprise a C2-C15 alkyl and/a C2-C15 glycol, for example a thioethyl group or a thiopropyl group.

The linker may be (i) HS—(CH₂)₂—CONH-AAY; (ii) HS—(CH₂)₂—CONH-LAAY; (iii) HS—(CH₂)₃—CONH-AAY; (iv) HS—(CH₂)₃—CONH— FLAAY; (v) HS—(CH₂)₁₀—(CH₂OCH₂)₇—CONH-AAY; and (vi) HS—(CH₂)₁₀—(CH₂OCH₂)₇—CONH-FLAAY. In this case, the thiol group of the non-peptide portion of the linker links the linker to the core.

Other suitable linkers for attaching a peptide to a nanoparticle are known in the art, and may be readily identified and implemented by the skilled person.

As explained above, the vaccine composition may comprise multiple filovirus peptides each comprising one or more of the CD8+ T cell epitopes set out in SEQ ID NOs: 1 to 9. The vaccine composition may comprise one or more additional peptides each comprising an epitope, such as a CD4+ T cell epitope, a B cell epitope, or a CD8+ T cell epitope other than the CD8+ T cell epitopes set out in SEQ ID NOs: 1 to 9. Thus, the vaccine composition may comprise more than one peptide.

When the vaccine composition comprises more than one peptide, two or more (such as three or more, four or more, five or more, ten or more, or twenty or more) of the peptides may be attached to the same nanoparticle. Two or more (such as three or more, four or more, five or more, ten or more, or twenty or more) of the peptides may each be attached to different nanoparticle. The nanoparticles to which the peptides are attached may though be the same type of nanoparticle. For instance, each peptide may be attached to a gold nanoparticle. Each peptide may be attached to a CaP nanoparticle. The nanoparticle to which the peptides are attached may be a different type of nanoparticle. For instance, one peptide may be attached to a gold nanoparticle, and another peptide may be attached to a CaP nanoparticle.

### Medicaments, Methods and Therapeutic Use

The invention provides a method of preventing or treating a filovirus infection, comprising administering the vaccine composition of the inventions to an individual infected with, or at risk of being infected with, a filovirus. The invention also provides a vaccine composition of the invention for use in a method of preventing or treating a filovirus infection in an individual.

The filovirus infection may be, for example, an ebolavirus infection or marburgvirus infection. The ebolavirus infection may be, for instance, a Zaire ebolavirus (ZEBOV), Sudan ebolavirus (SUDV), Reston ebolavirus (RESTV), Taï Forest ebolavirus (TAFV) or Bundibugyo ebolavirus (BDBV) infection. The marburgvirus infection may be, for instance, a Marburg virus (MARV) or Ravn virus (RAVV) infection

The vaccine composition may be provided as a pharmaceutical composition. The pharmaceutical composition preferably comprises a pharmaceutically acceptable carrier or diluent. The pharmaceutical composition may be formulated using any suitable method. Formulation of cells with standard pharmaceutically acceptable carriers and/or excipients may be carried out using routine methods in the pharmaceutical art. The exact nature of a formulation will depend upon several factors including the cells to be administered and the desired route of administration. Suitable types of formulation are fully described in Remington's Pharmaceutical Sciences, 19th Edition, Mack Publishing Company, Eastern Pennsylvania, USA.

The vaccine composition or pharmaceutical composition may be administered by any route. Suitable routes include, but are not limited to, the intravenous, intramuscular, intraperitoneal, subcutaneous, intradermal, transdermal and oral/buccal routes.

Compositions may be prepared together with a physiologically acceptable carrier or diluent. Typically, such compositions are prepared as liquid suspensions of peptide-linked nanoparticles. The nanoparticles may be mixed with an excipient which is pharmaceutically acceptable and compatible with the active ingredient. Suitable excipients are, for example, water, saline, dextrose, glycerol, of the like and combinations thereof.

In addition, if desired, the pharmaceutical compositions may contain minor amounts of auxiliary substances such as wetting or emulsifying agents, and/or pH buffering agents.

The peptide-linked nanoparticles are administered in a manner compatible with the dosage formulation and in such amount will be therapeutically effective. The quantity to be administered depends on the subject to be treated, the disease to be treated, and the capacity of the subject's immune system. Precise amounts of nanoparticles required to be administered may depend on the judgement of the practitioner and may be peculiar to each subject.

Any suitable number of nanoparticles may be administered to a subject. For example, at least, or about, 0.2×10⁶, 0.25×10⁶, 0.5×10⁶, 1.5×10⁶, 4.0×10⁶ or 5.0×10⁶ nanoparticles per kg of patient may administered. For example, at least, or about, 10⁵, 10⁶, 10⁷, 10⁸, 10⁹ nanoparticles may be administered. As a guide, the number of nanoparticles of the invention to be administered may be from 10⁵ to 10⁹, preferably from 10⁶ to 10⁸.

It is to be understood that different applications of the disclosed products and methods may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural referents unless the content clearly dictates otherwise. Thus, for example, reference to “a peptide” includes “peptides”, reference to “a nanoparticle” includes two or more such nanoparticles, and the like.

All publications, patents and patent applications cited herein, whether supra or infra, are hereby incorporated by reference in their entirety.

The following Examples illustrate the invention.

### Example 1

### INTRODUCTION

In WO 2012/050193, 94 HLA-A2 and 27 HLA-A24 ninemer MHC-1 peptide epitopes were identified using a search of the Ebola Zaire polyprotein. HLA binding was confirmed experimentally. Using A24 Tg and Human leukocyte antigen (HLA)-A2.1 transgenic (HHD) mouse models, 14 HLA-A2 and nine HLA-A24 peptides were confirmed to have strong in vivo Ebola virus-specific CTL cell inducing ability. The ability to induce CTL was assessed by increased proportion of CD8+/IFN-γ+T cells. Cross-reactivity between certain ebolavirus strains was established by assessing epitope conservation between strains. The present inventors have expanded upon this work by comprehensively investigating cross-reactivity between all ebolavirus strains and between Marburg virus. Cross-reactivity was determined using a BLAST analysis by comparing each peptide sequences (derived from the Ebola Zaire genome) to the genome of all other ebolavirus strains and of Marburg virus, and assessing sequence homology indicative of MHC-1 binding.

Of the peptides confirmed to have CTL inducing ability, six HLA-A2 peptides and three HLA-A24 peptides were selected by the present inventors as lead candidates based on strength of CTL induction and Filovirus cross-reactivity (Table 2). Two of the A2 peptides were subsequently shown to have HLA-24 cross binding. The four A2 peptides and two A2/A24 peptides (Table 3) peptides were verified in in vitro experiments where human blood donated from naïve donors (i.e. healthy donors who have not been previously infected by Ebola) was tested for a primary immune response to the peptide epitopes. These experiments mimic the process of immunisation at a cellular level and provide proof of mechanism that the peptide epitopes are able to immunise a naïve cell.

TABLE 2

| SEQ | | | | |
|---|---|---|---|---|
| ID | | Protein | HLA | Conserved |
| NO: | Sequence | ID | affinity | between |
| 1 | KIIKFLEPL | L-293 | a2/24 | EBOV, SUDV, |
| | | | | BDBV, RESTV, |
| | | | | TAFV, MARV |
| 2 | GLFQLKTYL | L-932 | a2 | EBOV, SUDV, |
| | | | | BDBV, RESTV, |
| | | | | TAFV |
| 3 | VLKAVVLKV | L-1955 | a2 | EBOV, SUDV, |
| | | | | BDBV, RESTV, |
| | | | | TAFV |
| 4 | RLAKLTEAI | NP-401 | a2 | EBOV, SUDV, |
| | | | | BDBV, RESTV, |
| | | | | TAFV |
| 5 | IIQAFEAGV | NP-56 | a2 | EBOV, SUDV, |
| | | | | BDBV, RESTV, |
| | | | | TAFV |
| 6 | KYTMQDALF | L-105 | a24 | EBOV, SUDV, |
| | | | | RESTV, MARV |
| 7 | KYQVKTLFF | L-1847 | a24 | EBOV, SUDV, |
| | | | | BDBV, RESTV, |
| | | | | TAFV |
| 8 | QYADCELHL | L-2046 | a24 | EBOV, BDBV, |
| | | | | TAFV, MARV |
| 9 | SLTDRELLL | VP30-94 | a2/24 | EBOV, SUDV, |
| | | | | BDBV, RESTV, |
| | | | | TAFV, MARV |

TABLE 3
A2 and A2/A24 peptides verified in
CTL experiments.

| Notation | Sequence | Affinity | Virus |
|---|---|---|---|
| P1 | KIIKFLEPL | A2/24 | EBOV, SUDV, BDBV, |
| | | | RESTV, TAFV, MARV |
| P2 | GLFQLKTYL | A2 | EBOV, SUDV, BDBV, |
| | | | RESTV, TAFV |
| P3 | VLKAVVLKV | A2 | EBOV, SUDV, BDBV, |
| | | | RESTV, TAFV |
| P4 | RLAKLTEAI | A2 | EBOV, SUDV, BDBV, |
| | | | RESTV, TAFV |
| P5 | IIQAFEAGV | A2 | EBOV, SUDV, BDBV, |
| | | | RESTV, TAFV |
| P6 | SLTDRELLL | A2/24 | EBOV, SUDV, BDBV, |
| | | | RESTV, TAFV, MARV |
| P7 | KYTMQDALF | a24 | EBOV, SUDV, RESTV, |
| | | | MARV |
| P8 | KYQVKTLFF | a24 | EBOV, SUDV, BDBV, |
| | | | RESTV, TAFV |
| P9 | QYADCELHL | a24 | EBOV, BDBV, TAFV, |
| | | | MARV |

### Methods

Epitope-specific CTLs were generated using peripheral blood mononuclear cells from healthy (naive) human HLA-A2+ donor. First, DCs were generated from an adherent population of PBMCs cultured in GM-CSF- and IL-4-containing medium. DCs obtained by this method (immature DCs) were pulsed with peptides (P1 to P6, individually) or nanoparticle-conjugate peptides (P1 to _6, individually). Both groups were supplemented with microglobulin. CD8+ T-cells were co-cultured with DCs at a ratio of 20:1 CD8+ T-cells to DCs in complete RPMI supplemented with 10% FBS and recombinant human IL-7 in 24 well plates. After three to four rounds of restimulation, cultures were analysed for CTL response.

Activated T-cells generated in accordance with this method were tested for cytotoxic activity against filovirus peptide-loaded T2 cells, and filovirus-infected HepG2 cells as targets in the following assays: production of IFN-γ and granzyme-B by ELISpot; cytokine secretion by MAGPIX assay; CD107a co-expression by flow cytometry.

### Stimulation of CTL Responses In Vitro

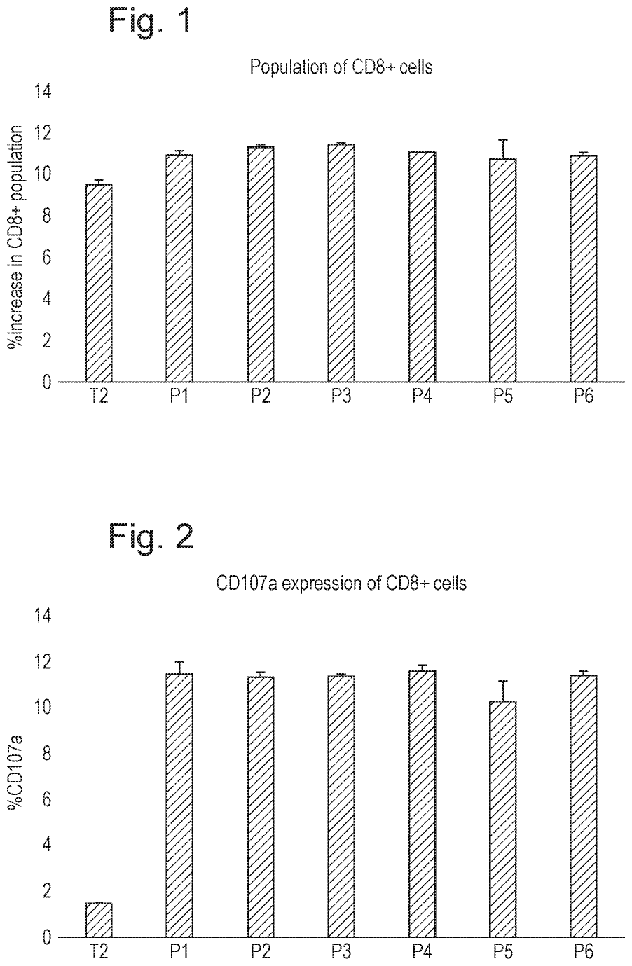

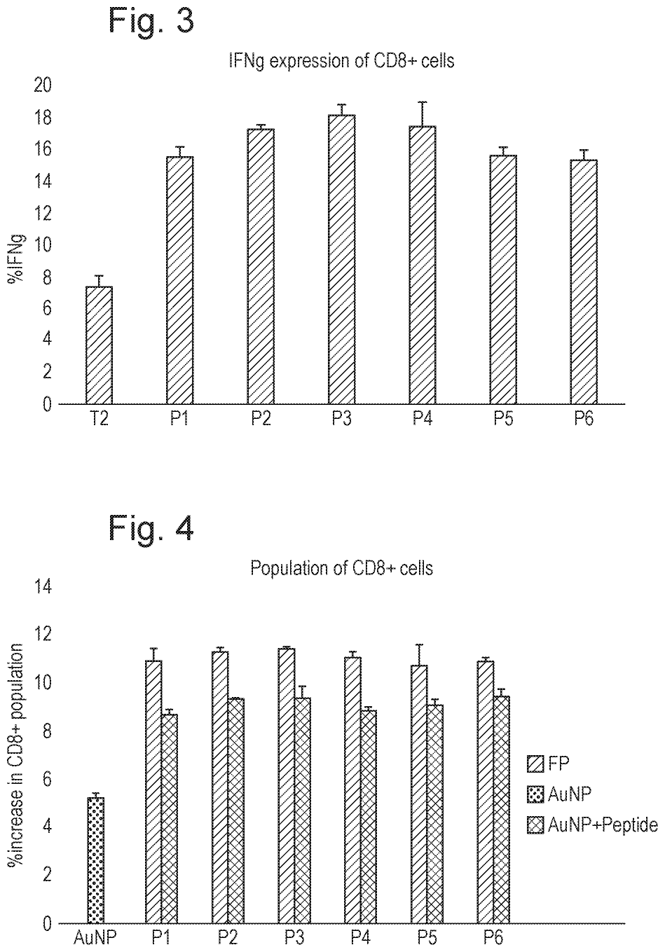

Utilizing a healthy (naive) human HLA-A2+ donor, peripheral blood mononuclear cells (PBMCs) were stimulated with peptide epitopes (P1 to P6) in a cytokine cocktail to induce antigen specific CTL response. These stimulated PBMCs were then assayed by co-culturing peptide loaded targets for antigen specific response. TAP-deficient cells (T2) were used for peptide loading, and blank T2 cells used as control. Expanded PBMCs (FIG. 2) were assayed for both CD107a degranulation (FIG. 1) and interferon gamma (IFN-g) (FIG. 3) markers by flow cytometry. All 6 peptide (P1-6) epitopes induced CD8+CD107a and IFNγ expression to peptide loaded T2 cells in a Filovirus peptide specific manner. For CD8+ expansion there was a high background of non-peptide specific expansion. This likely to be due to spill-over of excess peptides from the initial peptide stimulation stage loading T2 cells.

Naïve PMBC donor studies have also been used to compare to the free peptide form of P1 to P6 with P1 to P6 individually conjugated to a gold nanoparticle (NP) delivery system (AuNP+peptide). The AuNP used in these studies was a gold nanoparticle comprising a passivating surface of alpha-galactose and/beta-GlcNHAc. These experiments illustrate whether the peptide epitopes can be cleaved from the AuNP-conjugate delivery system.

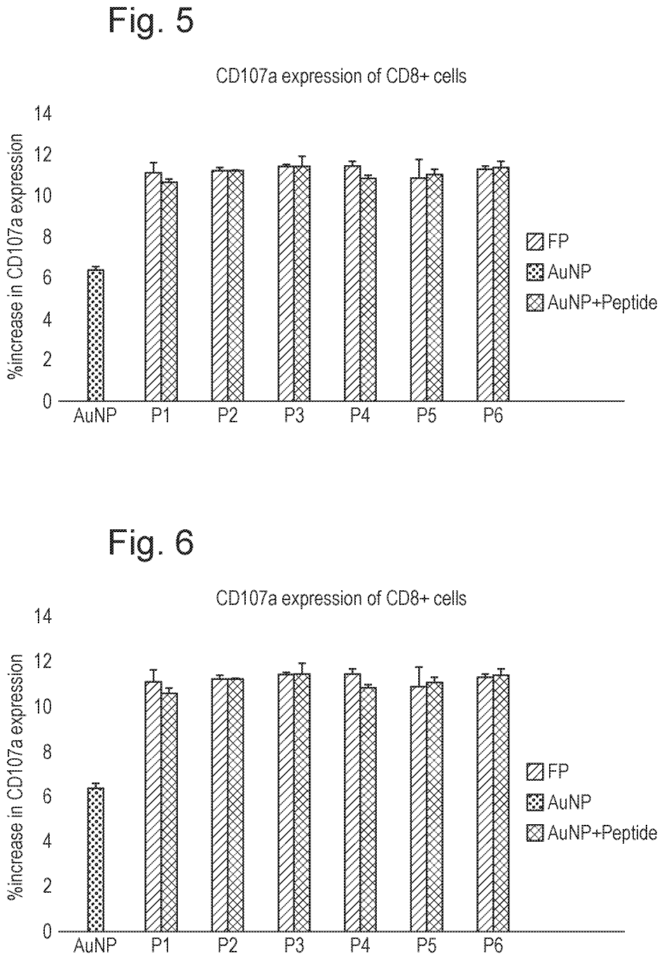

In more detail, utilizing a healthy (naive) human HLA-A2+ donor, peripheral blood mononuclear cells (PBMCs) were stimulated with peptide epitopes (P1 to P6) and individual AuNP-peptide conjugates thereof in a cytokine cocktail to induce antigen specific CTL response. These stimulated PBMCs were then assayed by co-culturing peptide loaded targets for antigen specific response. TAP-deficient cells (T2) were used for peptide loading, and blank T2 cells used as control. Expanded PBMCs (FIG. 4) were assayed for both CD107a degranulation (FIG. 5) and interferon gamma (IFN-g) (FIG. 6) markers by flow cytometry. The immune-stimulating capability of the AuNP base particle was also assessed in the absence of peptide to determine the strength of the base particle adjuvant signal. All AuNP-peptide conjugates had significant CTL inducing activity, which was moderately lower than free peptide in come cases and equivalent or superior in other cases depending on the CTL marker being assessed.

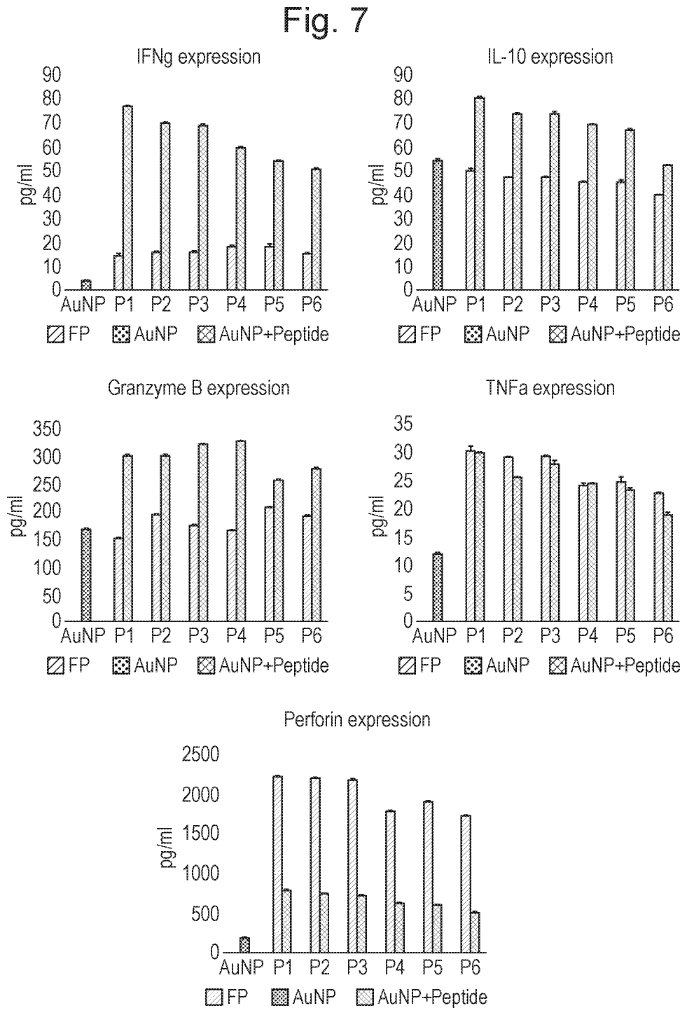

In addition to flow cytometric analysis of the PBMCs, the supernatant from assay cultures was collected for cytokine expression characterization. Multiple cytokines were analysed simultaneously using the multiplex capability of the Luminex MAGPIX system. Expressed cytokines from supernatants of activated PBMCs P1 to P6 and activated control (T2) supernatant of PBMCs are shown in FIG. 7.

### Example 2

### Methods

Epitope-specific CTLs were generated using peripheral blood mononuclear cells from healthy (naive) human HLA-A24+ donor. First, DCs were generated from an adherent population of PBMCs cultured in GM-CSF- and IL-4-containing medium. DCs obtained by this method (immature DCs) were pulsed with nanoparticle-conjugated peptides (P1, P6, P7, P8 and P9 individually) or a pool of nanoparticle-conjugated peptides (containing all of P1, P6, P7, P8 and P9). The nanoparticle used in this study was a gold nanoparticle comprising a passivating surface of alpha-galactose and beta-GlcNHAc. Medium was supplemented with microglobulin. CD8+ T-cells were co-cultured with DCs at a ratio of 20:1 CD8+ T-cells to DCs in complete RPMI supplemented with 10% FBS and recombinant human IL-7 in 24 well plates. After three to four rounds of restimulation, cultures were analysed for CTL response.

Activated T-cells generated in accordance with this method were tested for cytotoxic activity against filovirus peptide-loaded HepG2 cells as by analysing CD107a co-expression by flow cytometry.

### Stimulation of CTL Responses In Vitro

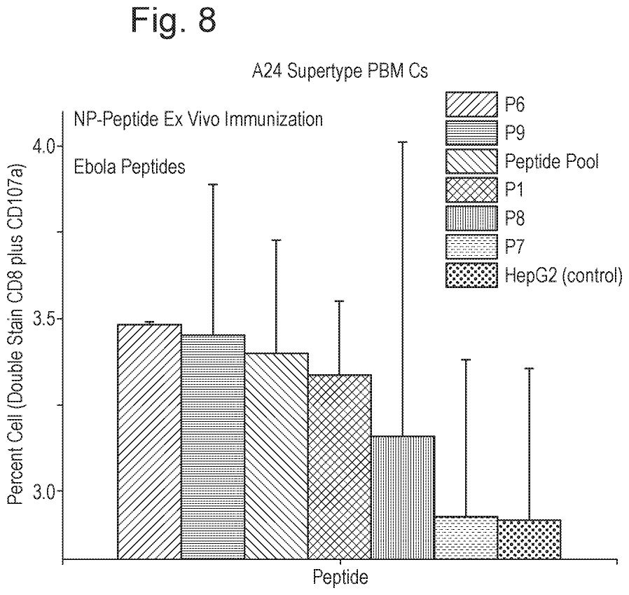

Utilizing a healthy (naive) human HLA-A2+ donor, peripheral blood mononuclear cells (PBMCs) were stimulated with peptide epitopes (P1, P6, P7, P8 and P9) in a cytokine cocktail to induce antigen specific CTL response. These stimulated PBMCs were then assayed by co-culturing peptide loaded targets for antigen specific response. HepG2 cells were used for peptide loading, and blank HepG2 cells used as control. Expanded PBMCs were assayed for expression of CD8 and CD107a (FIG. 8). Nanoparticle-conjugated P1, nanoparticle-conjugated P6, nanoparticle-conjugated P8, nanoparticle-conjugated P9, and pooled nanoparticle-conjugated P1, P6, P7, P8 and P9 all induced CD8+CD107a expression to peptide loaded HepG2 cells in a Filovirus peptide specific manner.

### Example 3

### Stimulation of CTL Responses In Vitro

Utilizing a healthy (naive) human HLA-A24+ donor, peripheral blood mononuclear cells (PBMCs) are stimulated with peptide epitopes (P1 to P6) in a cytokine cocktail to induce antigen specific CTL response. These stimulated PBMCs are then assayed by co-culturing peptide loaded targets for antigen specific response. TAP-deficient cells (T2) are used for peptide loading, and blank T2 cells used as control. Expanded PBMCs are assayed for both CD107a degranulation and interferon gamma (IFN-g) markers by flow cytometry.

In addition, naïve PMBC donor studies are used to compare to the free peptide form of P1 to P6 with P1 to P6 individually conjugated to a gold nanoparticle (NP) delivery system (AuNP+peptide). The AuNP used in these studies is a gold nanoparticle comprising a passivating surface of alpha-galactose and/beta-GlcNHAc. These experiments illustrate whether the peptide epitopes can be cleaved from the AuNP-conjugate delivery system.

In more detail, utilizing a healthy (naive) human HLA-A24+ donor, peripheral blood mononuclear cells (PBMCs) are stimulated with peptide epitopes (P1 to P6) and individual AuNP-peptide conjugates thereof in a cytokine cocktail to induce antigen specific CTL response. These stimulated PBMCs are then assayed by co-culturing peptide loaded targets for antigen specific response. TAP-deficient cells (T2) are used for peptide loading, and blank T2 cells used as control. Expanded PBMCs are assayed for both CD107a degranulation and interferon gamma (IFN-g) markers by flow cytometry. The immune-stimulating capability of the AuNP base particle is also assessed in the absence of peptide to determine the strength of the base particle adjuvant signal.

In addition to flow cytometric analysis of the PBMCs, the supernatant from assay cultures is collected for cytokine expression characterization. Multiple cytokines are analysed simultaneously using the multiplex capability of the Luminex MAGPIX system.

### Dextramer Studies

Dextramer reagents are fluorescently labelled and are used to detect antigen specific T-cells in cell suspensions and solid tissue samples. MHC dextramers are added to PBMCs or splenocytes. An optimal amount of anti-CD8 antibody conjugated with a relevant fluorochrome is then added. Additional antibodies (e.g. anti-CD3 or anti-CD4 antibodies) conjugated with other relevant fluorochromes may also be added at this step. Cells are then analysed using a flow cytometer.

### In Vivo CTL Studies

HLA A2/A24 transgenic mice (5-6 mice per group) are immunised with free synthetic peptide or nanoparticle-peptide conjugates mixed with and without montanide-51 adjuvant three times at 2-week intervals by subcutaneous and intra-dermal routes of administration. Spleen and draining lymph nodes are collected 7 days after the final boost for CTL analysis. Single cell suspensions are prepared from the lymphoid organs and cells are stimulated with peptide antigens in culture for 7 days. The reactivated T-cells are assayed for epitope specific CTL responses using filovirus peptide-loaded T2 cells and filovirus-infected HepG2 cells as targets in the following assays: production of IFN-γ and granzyme-B by ELISpot; cytokine secretion by MAGPIX assay; CD107a co-expression by flow cytometry.

### Adoptive Transfer Experiments

Adoptive transfer experiments are performed to investigate whether peptide-specific CTL generated in HLA-A2 transgenic mice have cytotoxic effect against Huh7 Ebola infected cells in vivo in SCID-Beige mice. Infected liver tumour suspension is injected sc or iv into SCID Beige mice, followed by single or multiple adoptive transfer of peptide specific CTL generated in transgenic A2 mice against peptide-NP constructs. Appropriate controls are used. Survival of mice is monitored.

1. A vaccine composition comprising a filovirus peptide comprising one or more of the CD8+ T cell epitopes set out in SEQ ID NOs: 1 to 9, wherein the peptide is attached to a nanoparticle.

2. The vaccine composition of claim 1, wherein the nanoparticle is a gold nanoparticle, a calcium phosphate nanoparticle, or a silicon nanoparticle.

3. The vaccine composition of claim 2, wherein the gold nanoparticle is coated with alpha-galactose and/or beta-GlcNHAc.

4. The vaccine composition of claim 1, wherein the filovirus peptide comprising a CD8+ T cell epitope is attached to the nanoparticle via a linker.

5. The vaccine composition of claim 1, which comprises two or more filovirus peptides each comprising a different CD8+ T cell epitope.

6. The vaccine composition of claim 5, wherein the two or more filovirus peptides are two or more of the peptides set out in SEQ ID NOs: 1 to 9.

7. The vaccine composition of claim 5 or 6, wherein each of the two or more filovirus peptides is attached to a nanoparticle.

8. The vaccine composition of claim 1, comprising at least two filovirus peptides comprising a CD8+ T cell epitope which each interacts with a different HLA supertype.

9. The vaccine composition of claim 1, which comprises at least one filovirus peptide comprising a CD8+ T cell epitope that interacts with at least two different HLA supertypes.

10. The vaccine composition of claim 8, wherein the at least two different HLA supertypes are selected from HLA-A1, HLA-A2, HLA-A3, HLA-A24, HLA-B7, HLA-B8, HLA-B27, HLA-B44, HLA-B58 and HLA-B62.

11. The vaccine composition of claim 10, wherein the at least two different HLA supertypes are HLA-A2 and HLA-A24.

12. The vaccine composition of claim 1, wherein the CD8+ T cell epitope is conserved between filoviruses.

13. The vaccine composition of claim 1, comprising a peptide comprising a CD4+ T cell epitope.

14. The vaccine composition of claim 13, wherein the CD4+ T cell epitopes interacts with all HLA class II types.

15. The vaccine composition of claim 13, wherein the CD4+ T cell epitope comprises the sequence set out in SEQ ID NO: 10 or 11.

16. The vaccine composition of claim 1, wherein the CD8+ T cell epitope is conserved between ebolaviruses and/or marburgviruses.

17. A method of preventing or treating a filovirus infection, comprising administering the vaccine composition of any one of the preceding claims to an individual infected with, or at risk of being infected with, a filovirus.

18. (canceled)

19. The method of claim 17, wherein the filovirus infection is an ebolavirus infection or marburgvirus infection.

20. The method of claim 19, wherein:
(a) the ebolavirus infection is a Zaire ebolavirus (ZEBOV), Sudan ebolavirus (SUDV), Reston ebolavirus (RESTV), Taï Forest ebolavirus (TAFV) or Bundibugyo ebolavirus (BDBV) infection; and/or
(b) the marburgvirus infection is a Marburg virus (MARV) or Ravn virus (RAVV) infection.


SEQUENCE LISTING (13 sequences)

SEQ ID NO: 1 - protein, 9 aa, Ebola virus
KIIKFLEPL

SEQ ID NO: 2 - protein, 9 aa, Ebola virus
GLFQLKTYL

SEQ ID NO: 3 - protein, 9 aa, Ebola virus
VLKAVVLKV

SEQ ID NO: 4 - protein, 9 aa, Ebola virus
RLAKLTEAI

SEQ ID NO: 5 - protein, 9 aa, Ebola virus
IIQAFEAGV

SEQ ID NO: 6 - protein, 9 aa, Ebola virus
KYTMQDALF

SEQ ID NO: 7 - protein, 9 aa, Ebola virus
KYQVKTLFF

SEQ ID NO: 8 - protein, 9 aa, Ebola virus
QYADCELHL

SEQ ID NO: 9 - protein, 9 aa, Ebola virus
SLTDRELLL

SEQ ID NO: 10 - protein, 18 aa, Clostridium tetani
FKLQTMVKLFNRIKNNVA

SEQ ID NO: 11 - protein, 19 aa, Clostridium tetani
LQTMVKLFNRIKNNVAGGC

SEQ ID NO: 12 - protein, 4 aa, Artificial Sequence
  Linker sequence
LAAY

SEQ ID NO: 13 - protein, 5 aa, Artificial Sequence
  Linker sequence
FLAAY